(19) United States
(12) Plant Patent Application Publication
(10) Pub. No.: US 2015/0342106 P1
(43) Pub. Date: Nov. 26, 2015

(54) Dianthus plant named 'KLEDP14206'

(50) ;

(71) Applicant: Klemm+Soln GmbH & Co. KG, Stuttgart (DE)

(72) Inventors: Nils Klemm, Stuttgart (DE); Martin Glawe, Stuttgart (DE)

(73) Assignee: Klemm+Soln GmbH & Co. KG, Stuttgart (DE)

(21) Appl. No.: 14/120,456

(22) Filed: May 20, 2014

(51) Int. Cl. A01H 5/00

(52) U.S. Cl. PLT/282

(57) ABSTRACT

A new Dianthus plant particularly distinguished by having double flowers with a dark pink, purple bicolor, and an upright plant habit, is disclosed.

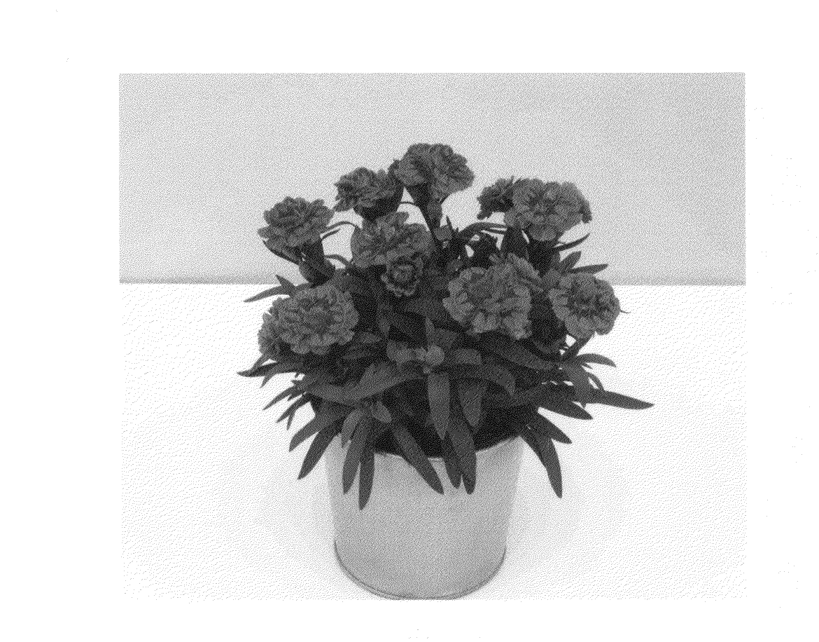

### GENUS AND SPECIES

Dianthus caryophyllus

### VARIETY DENOMINATION

‘KLEDP14206’

### BACKGROUND OF THE NEW PLANT

The present invention comprises a new and distinct variety of Dianthus, botanically known as Dianthus caryophyllus and hereinafter referred to by the variety name ‘KLEDP14206’. ‘KLEDP14206’ originated from a branch mutation of the proprietary female parent ‘DCP 2009-1003’ (unpatented) in May 2010 in Stuttgart, Germany.

In 2010 ‘KLEDP14206’ was first vegetatively propagated by vegetative cuttings. ‘KLEDP14206’ was found to reproduce true to type in successive generations of asexual propagation in Stuttgart, Germany via vegetative cuttings.

### SUMMARY

The following are the most outstanding and distinguishing characteristics of this new variety when grown under normal horticultural practices in Stuttgart, Germany.

1. Double flowers with a dark pink, purple bicolor; and

2. An upright plant habit.

### DESCRIPTION OF THE PHOTOGRAPH

This new Dianthus plant is illustrated by the accompanying photograph which shows an overall view of the plant. The colors shown are as true as can be reasonably obtained by conventional photographic procedures. The photograph shows the overall plant habit of the plant. The plant is about 5 months-old grown in Stuttgart, Germany in May 2012.

### DESCRIPTION OF THE NEW VARIETY

The following detailed descriptions set forth the distinctive characteristics of ‘KLEDP14206’. The data which define these characteristics were collected from asexual reproductions carried out in Stuttgart, Germany. Data was collected on five month-old plants grown in a greenhouse in Stuttgart, Germany in February 2014. Color references are to the R.H.S. Colour Chart of The Royal Horticultural Society of London (R.H.S.), 5^{th} edition (2007).
Classification:
   Family.—Caryophyllaceae.
   Species.—Dianthus caryophyllus.
   Common name.—Pot carnation.
   Variety.—‘KLEDP14206’.
Parentage: Result of a branch mutation of the proprietary Dianthus plant ‘DCP 2009 1003’
Propagation:
   Type.—Vegetative cuttings.
   Time to produce a rooted young plant.—14 to 16 days.
   Root description.—White, fine.
   Rooting habit.—Lanceolate.
Plant description:
   Plant form.—Potted plant, upright.
   Plant height.—13.0 cm to 15.0 cm.
   Plant diameter or spread.—20.0 cm to 22.0 cm.
Lateral branches:
   Length.—8.0 to 10.0 cm.
   Diameter.—3.0 to 5.0 cm.
   Internode length.—0.6 cm to 0.8 cm.
   Node length.—0.3 cm.
   Node diameter.—0.3 cm.
   Texture.—Smooth.
   Color.—RHS N137B.
Foliage description:
   Arrangement.—Opposite.
   Length.—4.0 to 6.0 cm.
   Width.—0.6 cm to 0.8 cm.
   Shape.—Lanceolate.
   Apex.—Acute.
   Base.—Obtuse.
   Margin.—Entire.
   Texture.—Upper and lower surfaces: Smooth.
   Venation pattern.—Palmate.
   Color.—Immature leaves: Upper surface: RHS 138A Lower surface: RHS 147B Mature leaves: Upper surfaces: RHS N139A Lower surface: RHS N137A Venation: RHS N139A.
Flower description:
   Flower type and habit.—Double, round, small sprays.
   Fragrance.—Present.
   Natural flowering season.—Spring to Fall.
   Flower longevity.—10 to 12 days.
   Flower buds.—Length: 1.8 cm to 2.0 cm Diameter: 0.8 to 1.0 cm Shape: Elliptic Color: RHS 145A, RHS 144A and RHS 183A.
   Corolla.—Quantity of petals/arrangement: 15 to 20, arranged in several whorls Corolla height: 1.5 cm to 2.0 cm Petal length: 3.5 cm Petal width: 1.4 cm to 1.6 cm Petal shape: Spoon-shaped Petal apex: Obtuse Petal base: Acute Petal margin: Crenate Petal texture: Upper and lower surfaces: Smooth Petal color: Immature: Upper surface: RHS 53A and RHS 61C Lower surface: RHS 63B and RHS 56A Mature: Upper surface: RHS 59A and RHS 67C Lower surface: RHS 56B and RHS 70C Calyx: Quantity of sepals/arrangement: 5, fused Calyx height: 1.8 cm to 2.0 cm Calyx diameter, at the apex: 0.8 cm to 1.0 cm Sepal texture: Upper and lower surfaces: Smooth Sepal color: Upper surface: RHS N137C Lower surface: RHS 145B.
Peduncles:
   Length.—12.0 cm to 14.0 cm.
   Diameter.—0.2 cm.
   Strength.—Moderate.
   Texture.—Smooth.
   Color.—RHS 137A.
Reproductive organs:
   Stamens.—Quantity: 3 to 5 Filament length: 0.5 cm to 0.7 cm Filament color: RHS 155B Anther length: 0.1 cm Anther diameter: 0.05 cm Anther color: RHS 19B Pollen amount: Sparse.
   Pistils.—Quantity: 1 Pistil length: 0.1 cm Style length: 1.6 cm to 1.8 cm Style color: RHS 59A Stigma shape: Filamentous Stigma color: RHS 59A Ovary size: 0.4 cm to 0.6 cm Ovary shape: Oval Ovary color: RHS 145A Fruits/seeds: None observed.
Disease and insect/pest resistance: No special observations were made.

### COMPARISON WITH PARENTAL LINE AND KNOWN VARIETY

‘KLEDP14206’ is a distinct variety of Dianthus. ‘KLEDP14206’ is distinguished from its parent as described in Table 1:

TABLE 1
Comparison with Parental Line

| | Variety | Parent |
|---|---|---|
| Trait | ‘KLEDP14206’ | ‘DCP 2009-1003’ |
| Flower Color | Dark pink, purple bicolor | Pink, purple eye |

‘KLEDP14206’ is most similar to the commercial Dianthus plant named commercially known as SuperTrouper ‘Oscar’ (unpatented). Differences between the two varieties are described in Table 2:

TABLE 2
Comparison with Similar Variety

| | Variety | Commercial line |
|---|---|---|
| Trait | ‘KLEDP14206’ | SuperTrouper ‘Oscar’ |
| Flower Color | Dark pink, purple bicolor | Dark pink with a purple eye |

We claim:

1 A new and distinct variety of Dianthus plant named ‘KLEDP14206’ as shown and described herein.
